(19) United States
(12) Patent Application Publication
(10) Pub. No.: US 2023/0335281 A1
(43) Pub. Date: Oct. 19, 2023

(54) MACHINE LEARNING AND ARTIFICIAL INTELLIGENCE BASED TOOL FOR SCREENING EMOTIONAL AND MENTAL HEALTH FOR AN INDIVIDUAL OR A GROUP OR MASSES

(71) Applicant: Sandeep VOHRA, New Delhi (IN)

(72) Inventor: Sandeep VOHRA, New Delhi (IN)

(21) Appl. No.: 18/210,791

(22) Filed: Jun. 16, 2023

Related U.S. Application Data
(63) Continuation of application No. PCT/IB2022/059076, filed on Sep. 26, 2022.

(30) Foreign Application Priority Data
Sep. 28, 2021 (IN) 202111043914

(51) Int. Cl. G16H 50/20; G16H 10/20; G16H 40/20; G16H 15/00; G16H 20/70

(52) U.S. Cl. CPC G16H 50/20; G16H 10/20; G16H 15/00; G16H 20/70; G16H 40/20; G06V 40/174

(57) ABSTRACT

A method for screening mental health using a screening tool. The method includes providing a set of questions to a user/subject by rendering on a user device. The set of questions rendered on the user device are provided by a questionnaire module. Further receiving natural language reply to each question in the set of questions, wherein the natural language replies are analyzed using a natural language processing module. The method further includes assigning a weightage to each question in the set of questions using the natural language processing module. Further generating an Emotional Wellness Index (EWI) for the user, by an assessment module based on the weightage assigned. The method further includes generating a report based on EWI, wherein a report generation module renders the level of distress to the user based on EWI and specific to which domain.

### CROSS-REFERENCE TO RELATED APPLICATIONS AND PRIORITY

The present application claims priority from PCT/IB2022/059076 filed on Sep. 26, 2022 and Indian Patent Application No: 202111043914, filed on 28 Sep. 2021, each of which are incorporated herein by a reference.

### TECHNICAL FIELD

The present subject matter described herein, in general, relates to a screening tool for mental health for an individual, a group and the masses, more particularly to an AI based screening system configured to initially screen and subsequently deduces mental health of the user by AI based algorithm followed by connecting it with a human professional interface on a digital platform as and when required in a uniquely triaging manner.

### BACKGROUND

The subject matter discussed in the background section should not be assumed to be prior art merely because of its mention in the background section. Similarly, a problem mentioned in the background section or associated with the subject matter of the background section should not be assumed to have been previously recognized in the prior art. The subject matter in the background section merely represents different approaches, which in and of themselves may also correspond to implementations of the claimed technology.

National Alliance on Mental Illness (NAMI) has introduced many free and confidential community information and referral services, but there are just observational results and not the diagnosis. Further the Mental Health Assessment Tool developed by the Heartland National TB Centre relies on a series of questionnaires and observations that can be documented during the patient’s medical evaluation. This tool was developed to bring awareness to behaviors that may potentially disrupt the TB treatment regimen. Substance abuse and mental illness may complicate case management. Knowledge of these issues can help TB program staff anticipate problems. However, use of the screening techniques and ratings in this tool does not produce a diagnosis. Rather, the tools and scales can point toward significant behaviors that may require further assessment by a professional. Similarly, the present subject matter helps in screening individual, group &/or masses in the field of mental health which leads to automated triaging leading to referral to a mental health professional such as psychologist, psychiatrist any trained person depending on the degree of the problem.

Traditionally, the researchers routinely use two forms of structured assessments. Structured interviews, such as the Structured Clinical Interview for DSM-IV (SCID), include a question for every symptom for each disorder in the DSM, and are often considered the “gold standard” in psychiatric assessment (Basco et al., 2000; Kranzler, Kaddeti, Burleson, & Babor, 1995; Shear et al., 2000). On the other hand, rapid assessment instruments, such as the Patient Health Questionnaire. These types of assessments generally consist of a relatively small number of items that attempt to identify the most common psychiatric symptoms and disorders.

Existing screening tools do not offer insights into the specific causes of stress, or the relative levels of stress experienced by individuals. Understanding the causes and levels of stress is crucial for developing effective interventions and providing targeted support. These screening tools are designed to assess emotional well-being and mental health problems but lack the capability to determine the underlying reasons for stress and its comparative intensity across different individuals. Further, the current system lacks the capability to perform a comparative analysis between multiple users, hindering its ability to assess stress levels across individuals. Additionally, it does not support the grouping and mapping of screening analysis and results, which limits its usefulness in identifying patterns and trends among different user groups. Moreover, the existing system fails to accurately define the causes and measure the severity of stress within specific domains, impeding its effectiveness in providing targeted interventions.

These limitations present significant obstacles in understanding and addressing stress-related issues. Therefore, there is a pressing need to develop an enhanced system that can facilitate comparative analysis, enable group mapping of screening and results, and accurately identify stress causes and levels within their respective domains. Such a system would greatly improve our ability to analyze and address stress-related challenges, ultimately leading to more effective interventions and support for individuals and groups.

However, the present subject matter is screening for emotional & mental health problems in both adults and students and then referring them to an automated system with an AI & machine learning based algorithm which tells then whether one requires professional help or not in an individual report format as well as group report format indicating how many require help and how many do not require help. This process can be scaled up indefinitely for infinite number of people by increasing the number of IT resources & server load balancing automation focused as per the number of concomitant users.

Accurate diagnosis is often regarded as the first and most important step in treating any medical condition. Thus, these exists a long-standing need of a system and method for a machine learning and AI based system and a method for a screening of mental health not only by using assessment of natural language answers to a questionnaire, but also by assessment through calculating an Emotional Wellness Index (EWI), a concept conceived, and the term coined by inventor Dr. Sandeep Vohra. It is world’s first emotional & mental wellness AI based process machine akin to BP or sugar checking machine which gives the subject’s reading and based on that result the subsequent treatment is suggested or followed by a medical professional’s advice.

### SUMMARY

This summary is provided to introduce concepts related to a machine learning and Al based system and a method for a screening tool on mental health and the concepts are further described below in the detailed description. This summary is not intended to identify essential features of the claimed subject matter nor is it intended for use in determining or limiting the scope of the claimed subject matter.

In one embodiment, a machine learning and AI based system for a screening tool for mental health is disclosed herein. The system may comprise a processor, and a memory coupled to the processor. The memory may comprise a plurality of programmed instructions executed by the processor. The plurality of programmed instructions may comprise of a questionnaire module for setting a first set of questions to the user. Further, the plurality of programmed instructions may comprise of a Natural Language Processing (NLP) Module for receiving and processing the natural language answers to a first set of questionnaires. The plurality of programmed instructions may further comprise an Expressions Capturing module for capturing expressions of the user while answering to the questionnaire. Furthermore, the plurality of programmed instructions may comprise an AI Module configured to perform AI operations such as pattern recognition and prediction via the processor. The AI module is also configured to generate a second set of questionnaires based on the answers received for the first set of questionnaires. The plurality of programmed instructions may also comprise an Assessment Module for assessment of Emotional Wellness Index (EWI) which helps in assessing overall degree of emotional stress and areas from where stress is emanating via a color band generation. The Assessment module may also give weightage to at least one of the domains that generate the stress through assessment of natural language processed answers for the first set of questions. The plurality of programmed instructions may also comprise a report generation module to describe the level of distress to the user.

In another embodiment, a machine learning and AI based method for a screening tool for mental health is disclosed herein. The machine learning and AI based method may comprise step of providing a first set of questions, by a processor to the user. The method may further comprise step of receiving natural language answers and processing them through machine learning, and further setting a second set of questionnaires to the user based on the answers received using an AI based module. Further, the method may comprise step of giving weightage to at least one of the domains creating stress and assessing the EWI of the user, by the processor using an Assessment Module, wherein the EWI will decide about the mental health status of the user by assessing overall degree of emotional stress and areas from where stress is emanating via a color band generation. Furthermore, the method may comprise step of generating report, describing level of distress to the user. In an implementation of the present disclosure a method for screening mental health using a screening tool (101) is disclosed. The method comprises providing (Step 501) a set of questions to a user/subject by rendering on a user device (103). The set of questions rendered on the user device (103) are provided by a questionnaire module (205). Further receiving (Step 502) natural language reply to each question in the set of questions, wherein the natural language replies are analyzed using a natural language processing module (206). The method may further comprise assigning (Step 502) a weightage to each question in the set of questions using the natural language processing module (206). Further generating (Step 504) an Emotional Wellness Index (EWI) for the user, by an assessment module (209) based on the weightage assigned, wherein the Emotional Wellness Index (EWI) is generated for at least one domain selected from domestic life domain, situational/circumstantial domain, nature of the user domain, professional life domain, and clinical domain. The method as disclosed may comprise generating (Step 505) a report based on EWI, wherein a report generation module (210) renders the level of distress to the user based on EWI and specific to which domain.

In another implementation system (101) for screening of mental health using a screening tool is disclosed. The system (101) comprises a user device (103) configured to render a set of questions provided by the questionnaire module (205). Further a natural language processing module (206) is configured to quantify the replies by assigning a defined weightage. The system may further comprise an AI module (208) configured select questions from the questionnaire module (205) to form another set of questions based on the weightage assigned to each question. Further an expression capturing module (207) is configured to capture the facial and other behavioral expression of the user while replying to each of the questions. The system may further comprise assessment module (209) configured to generate an Emotional Wellness Index (EWI) for each of the specific domain including domestic life, situational circumstantial, nature of the user, professional life, and clinical, wherein the (EWI) is based on the weightage assigned to each reply by the natural language processing module (206) and the AI module (208). Further a report generation module (210) configured to render the level of distress to the user.

### BRIEF DESCRIPTION OF DRAWINGS

The detailed description is described with reference to the accompanying Figures. In the Figures, the left-most digit(s) of a reference number identifies the Figure in which the reference number first appears. The same numbers are used throughout the drawings to refer like features and components.

FIG. I illustrates a network implementation 100 of a machine learning and AI based system 101 for a screening tool for mental health, in accordance with an embodiment of the present subject matter.

FIG. 2 illustrates components of the system 101, in accordance with an embodiment of the present subject matter.

FIG. 3 illustrates, an exemplary embodiment of Assessment module 209 in a machine learning and AI based system for a screening tool for mental health, in accordance with the present subject matter.

FIG. 4 illustrates, an exemplary embodiment of report of calculated EWI in a screening tool for mental health, in accordance with the present subject matter.

FIG. 5 illustrates, a machine learning and AI based method 500 for a screening tool on mental health, in accordance with an embodiment of the present subject matter.

### DETAILED DESCRIPTION

Reference throughout the specification to “various embodiments,” “some embodiments,” “one embodiment,” or “an embodiment” means that a particular feature, structure, or characteristic described in connection with the embodiment is included in at least one embodiment. Thus, appearances of the phrases “in various embodiments,” “in some embodiments,” “in one embodiment,” or “in an embodiment” in places throughout the specification are not necessarily all referring to the same embodiment. Furthermore, the particular features, structures or characteristics may be combined in any suitable manner in one or more embodiments.

The present disclosure illustrates a system and method to screen mental health in a plurality of subjects. The system and method as disclosed enables screening of the mental health by accessing the application over a device like handheld smart phones or laptops, or via smart interactive devices likes Siri, Alexa, or device enabling virtual or augmented interactions.

The system is configured to provide a preset of questionnaire through a questionnaire module. The questionnaire module may relay a defined or pre-selected list of question to the subject. The questions may be rendered on the display of the smart device, or verbally relayed over the speaker or such similar technological interfaces. Further the responses to the selected list question can be replied by selecting preset of replies or replying in the natural language.

Referring now to FIG. 1, illustrates a network implementation 100 of a machine learning and Al based system 101 for a screening tool on mental health, in accordance with an embodiment of the present subject matter.

The system 101 may be implemented as a network of variety of computing systems, such as a laptop computer, a desktop computer, a notebook, a workstation, a mainframe computer, a server, a network server, and the like. In one embodiment, the system 101 may be configured to be installed at one or more places, such as but not limited to, airport, railway station, bus stop, shopping mall market, office, coffee shops, restaurants, club, movie theatre, library, school, college, residential society, public park, public place and the like. The system 101 may be accessed by multiple users through one or more user devices 103-1, 103-2...103-N, collectively referred to as user device 103 hereinafter. Further one or more person accessing the system 101 via the user device 103 may be referred to as user or subject. The user devices 103 accessed by the subjects may include, but are not limited to, a portable computer, a personal digital assistant, a handheld device, and a workstation. The user devices 103 are communicatively coupled to the system 101 through a network 102.

In one implementation, the network 102 may be a wireless network, a wired network or a combination thereof. The network 102 can be implemented as one of the different types of networks, such as intranet, local area network (LAN), wide area network (WAN), the internet, and the like. The network 102 may either be a dedicated network or a shared network. The shared network represents an association of the different types of networks that use a variety of protocols, for example, Hypertext Transfer Protocol (HTTP), Transmission Control Protocol/Internet Protocol (TCP/IP), Wireless Application Protocol (WAP), and the like, to communicate with one another. Further the network 102 may include a variety of network devices, including routers, bridges, servers, computing devices, storage devices, and the like.

The aforementioned devices may support communication over one or more types of networks in accordance with the described embodiments. For example, some computing devices and networks may support communications over a Wide Area Network (WAN), the Internet, a telephone network (e.g., analog, digital, POTS, PSTN, ISDN, xDSL), a mobile telephone network (e.g., CDMA, GSM, NDAC, TDMA, E-TDMA, NAMPS, WCDMA, CDMA-2000, UMTS, 3G, 4G), a radio network, a television network, a cable network, an optical network (e.g., PON), a satellite network (e.g., VSAT), a packet-switched network, a circuit-switched network, a public network, a private network, and/or other wired or wireless communications network configured to carry data. Computing devices and networks also may support wireless wide area network (WWAN) communications services including Internet access such as EV-DO, EV-DV, CDMA/1xRTT, GSM/GPRS, EDGE, HSDPA, HSUPA, and others.

The aforementioned devices and networks may support wireless local area network (WLAN) and/or wireless metropolitan area network (WMAN) data communications functionality in accordance with Institute of Electrical and Electronics Engineers (IEEE) standards, protocols, and variants such as IEEE 802.11 (“WiFi”), IEEE 802.16 (“WiMAX”), IEEE 802.20x (“Mobile-Fi”), and others. Computing devices and networks also may support short range communication such as a wireless personal area network (WPAN) communication, Bluetooth® data communication, infrared (IR) communication, near-field communication, electromagnetic induction (EMI) communication, passive or active RFID communication, micro-impulse radar (MIR), ultra-wide band (UWB) communication, automatic identification and data capture (AIDC) communication, and others.

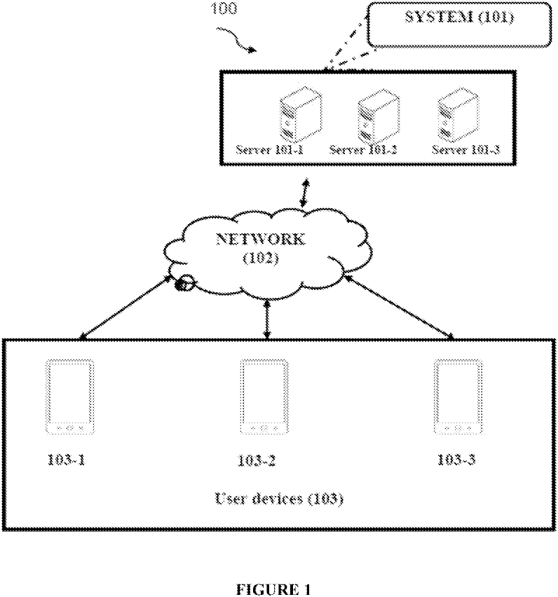

Referring now to FIG. 2, the system 101 is illustrated in accordance with an embodiment of the present subject matter. In one embodiment, the system 101 may include one processor 201, an input/output (I/O) interfaces 202, and one memory 203. The processor 201 may be implemented as microprocessors, microcomputers, microcontrollers, digital signal processors, central processing units, state machines, logic circuitries, and/or any devices that manipulate signals based on operational instructions. Among other capabilities, the processor 201 is configured to fetch and execute computer-readable instructions stored in the memory 203.

The I/O interface 202 may include a variety of software and hardware interfaces, for example, a web interface, a graphical user interface, and the like. The I/O interface 202 may allow the system 101 to interact with a user directly or through the client devices 103. Further, the I/O interface 202 may enable the system 101 to communicate with other computing devices, such as web servers and external data servers (not shown). The I/O interface 202 can facilitate multiple communications within a wide variety of networks and protocol types, including wired networks, for example, LAN, cable, etc., and wireless networks, such as WLAN, cellular, or satellite. The I/O interface 202 may include one or more ports for connecting a number of devices to one another or to another server.

The memory 203 may include any computer-readable medium known in the art including, for example, volatile memory, such as static random-access memory (SRAM) and dynamic random-access memory (DRAM), and/or non-volatile memory, such as read only memory (ROM), erasable programmable ROM, flash memories, hard disks, optical disks, and magnetic tapes. The memory 203 may include modules 204 and data 212.

The programmed instructions/modules 204 include routines, programs, objects, components, data structures, etc., which perform particular tasks or implement particular abstract data types. In one implementation, the modules 204 may include a questionnaire module 205, a natural language processing module 206, an expression capturing module 207, AI modules 208, Assessment Module 209 and Report generation Module 210. Further the modules 204 may include other modules 211, wherein the other modules 211 may include programs or coded instructions that supplement applications and functions of the system 101.

The data 212, amongst other things, serves as a repository for storing data processed, received, and generated by one or more of the modules 204. The data 212 may also include a network data 213 and other data 214. The other data 214 may include data generated as a result of the execution of one or more modules in the other module 211. In one implementation, at first, a user may use the user device 103 to access the system 101 via the I/O interface 202. The user may register themselves using the I/O interface 202 in order to access the system 101. The system 101 may be used for the real-time monitoring of network data in the questionnaire event. The processor 201 may be communicatively coupled with a plurality of external systems. In one embodiment, the usage of screening tool for mental health may be indicative of activities or operations or functionalities performed by one or more external systems for executing diagnosis process e.g., process workflow) in the network. In one exemplary embodiment, the plurality of external systems may include, but not limited to, a Machine learning and AI based system, a natural language processing system, an expression capturing system, an Assessment system, and a report generation system. In one embodiment, the processor 201 of the machine learning and AI based system 101 may be configured for configuring and storing one or more predefined data. In one exemplary embodiment, the one or more predefined data may comprise a sequence of instructions which should be performed for network data while usage of the screening tool on mental health.

In accordance with an exemplary embodiment, of the present disclosure the user, or subject, or plurality of subjects may access the screening tool on mental health either simultaneously (for community screening) or alternately (for individual screen). The screening tool embedded in the system 101 may be accessed by the users or subjects via the user device 103. In an exemplary embodiment the screening tool may be partially embedded in the user device 103 and partially in the system 101 placed remotely.

The user device 103 as illustrated above may be handheld device, or an interactive device like smart glasses, Alexa or Siri or Google, or interactive platforms like metaverse or any such virtual interactive platforms such as, but not limited to, chatbot, personal assistant, voice assistant, Chat-GPT, simulation, metaverse, omniverse, virtual reality (VR), mixed reality (MR), augmented reality (AR), eXtended Reality (XR), and other futuristic technical development. In one embodiment, the user device 103 may comprise, but not limited to, self-service kiosks, Automated Teller Machines (ATMs), digital booth, vending machines, Dispensing machine, point of sale (POS), Touchscreen terminal, interactive whiteboards, smartboards, Interactive displays, self-checkout machines, and other self-service machines and the like The user devices 103 may be configured to render a set of questions provided by the questionnaire module 205. Further the replies to the set of questions may be captured and analyzed by a natural language processing module 206 to quantify the replies by assigning a defined weightage. Based on the weightage assigned to each question, an AI module 208 may determine or select questions from the questionnaire module 205 to form another set of questions. The another set of questions may be rendered on the user device 103 via the questionnaire module 205. Further replies to the another set of question is analyzed by the natural language processing module 206 to quantify the replies by assigning a defined weightage.

In accordance with the disclosure the process, of selecting questions by the AI module 208, from the questions in the questionnaire module 205, based on the weightage assigned to previous set of questions by the natural language processing module 206, is repeated/reiterated until questions about domestic life, situational/circumstantial, nature of the user, professional life, and clinical questions are covered. Further an expression capturing module 207 is configured to capture the facial and other behavioral expression of the user while replying to each of the questions. The AI module 208, may be configured further to amend the weightage assigned to each reply based on captured expression. The expression may be captured using I/O interface 202. The I/O interface 202 may comprise camera, and/or mic to capture the expression and change in voice tone.

Based on the weightage assigned to each reply by the natural language processing module 206 and the AI module 208, the assessment module 209 may be configured to generate an Emotional Wellness Index (EWI) for each of the specific domain including domestic life, situational/circumstantial, nature of the user, professional life, and clinical. Further based on EWI, the report generation module 210 may render the level of distress to the user. Further incorporating with advanced algorithms and techniques to define and measure the causes and levels of stress within respective domains accurately. This involves leveraging natural language processing, machine learning, and data analytics to identify key stressors and their impact on individuals in specific contexts. The report generation module 210 may render varying levels of distress related to specific domains, indicating the extent of distress in relation to the underlying causes. Further based on the distress level of the user and in which specific domain, the system 101 may be configured to recommend the user an AI based therapy, or human interaction-based therapy for the reducing the stress. The human interaction-based therapy may be conducted by trained health-care professionals, comprise a community of psychologists, psychiatrists, or any trained person, providing mental and behavioral health professional services worldwide.

In another exemplary embodiment the user device 103 may be comprise interactive devices or interactive platforms such as metaverse. The interactive devices may interact with the user in real-time by relaying questions from the questionnaire module 205 in conversational way using combination of speaker, and/or mic, and/or camera. Similar to relaying the questions, the replies to the questions may be captured in conversational way, and analyzed by the natural language processing module 206. The natural language processing module 206 may quantify and standardize the replies by assigning weightage to each reply. The AI module 208 in accordance with the exemplary embodiment may be configured to amend the weightage assigned to the reply based on the expression captured by the expression capturing module 207, and further select set of questions based on the weightage to previous set of questions. The questions are selected from the questionnaire module 205 and further relayed on the interactive user device 103.

The above process of relaying question, assigning weightage, selecting questions, and again replaying question is repeated/reiterated until questions about domestic life domain, situational/circumstantial domain, nature of the user domain, professional life domain, and clinical domain are covered. The expression capturing module 207 is configured to capture the facial and other behavioral expression of the user while replying to each of the questions including change in voice tone while the question relaying domestic life domain, situational/circumstantial domain, nature of the user domain, professional life domain, and clinical domain are covered.

Further in accordance with the exemplary embodiment, based on the weightage assigned to each reply by the natural language processing module 206 and the AI module 208, the assessment module 209 may be configured to generate an Emotional Wellness Index (EWI) for each of the specific domain including domestic life, situational/circumstantial, nature of the user, professional life, and clinical. Further based on EWI, the report generation module 210 may render the level of distress to the user and providing the user with a clear understanding of their level of distress within a particular domain. By specifying the domain, the report effectively communicates the areas where the user may be experiencing heightened emotional difficulties or challenges.

Further based on the distress level of the user and in which specific domain, the system 101 may be configured to recommend the user an AI based therapy, or human interaction-based therapy for the reducing the stress. The human interaction-based therapy may be conducted by trained health-care professional.

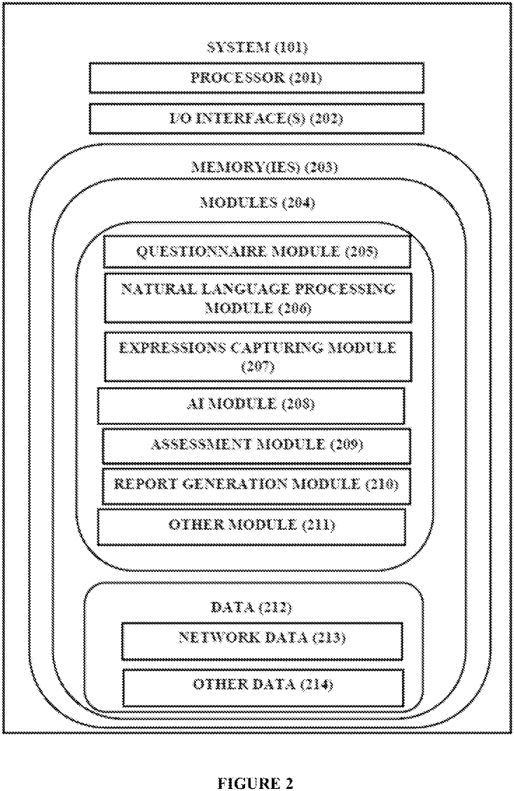

Now referring to FIG. 3, illustrates domains of Emotional Wellness Index (EWI), in accordance with an exemplary embodiment of the present subject matter. An assessment module 209 of the system 101 is configured to generate an Emotional Wellness Index (EWI) for domestic life domain, situational/circumstantial domain, nature of the user domain, professional life domain, and clinical domain. The assessment module 209 is configured to generate EWI based on the weightage assigned by a natural language processing module 206 and an AI module 208 to each question covering the above-mentioned domains. The EWI generated by the assessment module 209 in accordance with the exemplary embodiment may comprise and show cumulated weightage for each domain, based on summation of weightage assigned to questions covering a specific domain.

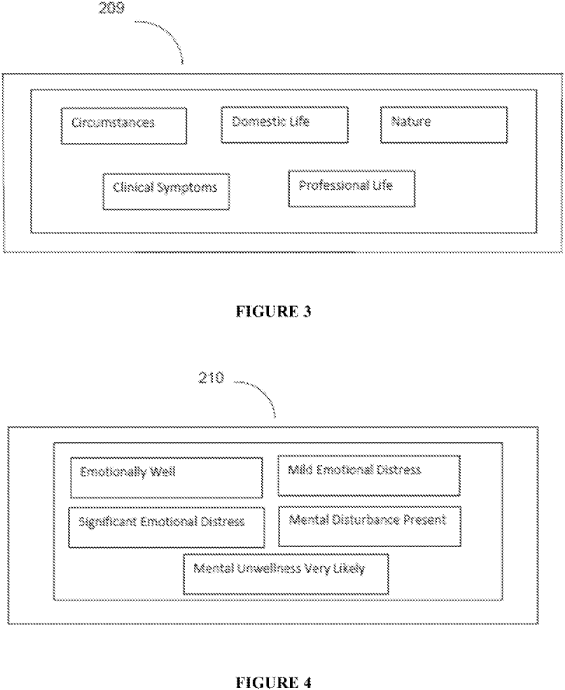

Now, referring to FIG. 4, concept of EWI is depicted which may ultimately be reflected in the report using the report generation module 210, in accordance with a first exemplary embodiment of the present subject matter.

In one exemplary embodiment, the report generation module 210, may render the level of distress to the user, wherein the “NORMAL” level of distress may be shown in Green color depicting that the user may be in a “Emotionally Well” state; the “MILD” level of distress may be shown in Blue color depicts that the user may be in “Mild Emotional Distress” state; the “MODERATE” level of distress shown in Yellow color depicts that the user may be in “Significant Emotional Distress” state; the “SEVERE” level of distress shown in Orange color depicts that the user may be in “Mental Disturbance Present” state; the “PROFOUND” level of distress shown in Red color depicts that the user may be in “Mental Unwellness Very Likely” state.

In another embodiment, the report generation module 210, may render to the user different levels of distress related to specific domains, indicating extent of distress in relation to the underlying causes. In an exemplary embodiment, the report generation module 210 may render to the user, one or more domains such as domestic life domain, situational/circumstantial domain, nature of the user domain, professional life domain, and clinical domain, along with corresponding level of distress from the one of “NORMAL”, “MILD”, “MODERATE”, “SEVERE” and “PROFOUND” for each domain. The report generation module 210 may also render different color bands among the (Green, Blue, Yellow, Orange and Red) indicating the level of distress corresponding to each domain.

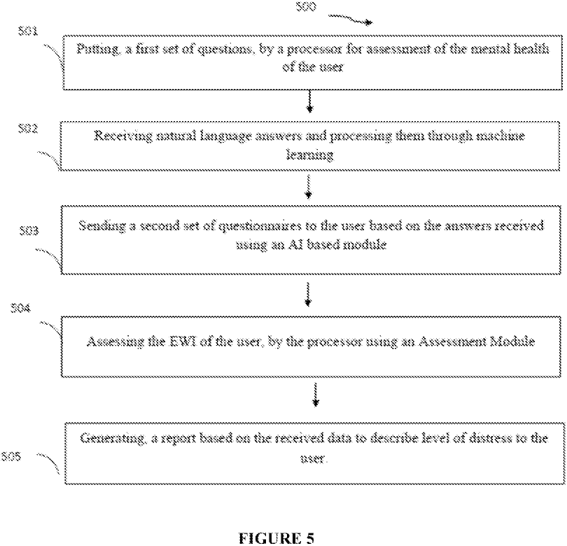

Now referring to FIG. 5, a method for screening tool on mental health is illustrated. At step 501, the processor 201 may be configured to provide a set of questions to a user/subject on user device 103. The set of questions can be rendered on the user device 103 or relayed as a conversation with the user. Further a questionnaire module 205 may be configured to provide the set of questions.

At step 502, the processor 201 may be configured to receive natural language reply to each question in the set of questions. The natural language replies may be analyzed using a natural language processing module 206 to quantify the replies by assigning a weightage. Further the assigned weightage may be amended by an Artificial Intelligence (AI) module 208. The weightage may be amended by the AI module 208 based on the expression captured by an expression capturing module 207. Facial and behavioral expression may be captured while replying to each question, and based on the facial and behavioral expression captured by the expression capturing module 207 the weightage may be amended by the AI module 208. The expression capturing module 207 may configured to capture the facial expression of the lips, eyes and change in tone of the voice.

At step 503, the processor 201 may be configured for sending another set of questions to the user. Based on the weightage assigned to each question, in the previous set of questions, an AI module 208 may determine or select questions from the questionnaire module 205 to form another set of questions. The AI module 208 may be further configured to update the questions in the questionnaire module 205 via machine learning. The update of the questions may be based on the historical data, and number of subjects or users being screened using the screening tool. Further in accordance with an exemplary embodiment as the data set increases, i.e., upon screening a sizable number of users/subjects the questions can be updated to reflect categories like profession, or geography, or sector, age group and such.

Further steps 501 to 503 are repeated until questions about domestic life domain, situational/circumstantial domain, nature of the user domain, professional life domain, and clinical questions domain are covered.

At step 504, the processor 201 may be configured for generating the EWI of the user, by the processor using an assessment Module 209. The EWI generated by the assessment module 209 for each of the specific domain including domestic life, situational/circumstantial, nature of the user, professional life, and clinical is based on the weightage assigned to each reply by the natural language processing module 206 and the AI module 208.

At step 505, the processor 201 may be configured for generating a report based on EWI. A report generation module 210 may render the level of distress to the user based on EWI. The report generation module 210 plays an active role in creating a detailed report that utilizes the Emotional Wellness Index (EWI) acquired. This report aims to provide the user with a deep understanding of their level of distress in a particular area. By identifying the specific domain, the report effectively communicates the precise areas where the user might be facing increased emotional struggles or obstacles, thereby empowering them with valuable insights. Further based on the distress level of the user and in which specific domain, the system 101 may be configured to recommend the user an Al based therapy, or human interaction-based therapy for the reducing the stress. Moreover, the report goes beyond just highlighting the domains and provides valuable insights by showcasing the level of distress with respect to the causes of distress. In cases where the user is facing multiple stress-related issues, the report accurately displays the levels of distress associated with each cause. This way, the user gains a deep understanding of the different factors contributing to their overall emotional well-being and can identify the specific areas that require attention and support. The report serves as a meaningful tool for self-reflection and empowers the user to take appropriate steps towards addressing their emotional wellness. The human interaction-based therapy may be conducted by trained health-care professional.

Further the system 101 may be configured for automated triaging, by referring users to one of, psychologist, psychiatrist, any trained person or a combination thereof, after a screening process. This may offer both direct appointment-based services as well as digital screening and referral services. The automated triaging may help to streamline the process of accessing mental health services and ensure that users receive the appropriate level of care based on their needs. Further, the system 101 may provide a single platform for specific user-based digital interaction amongst psychologists, psychiatrists or any trained person, to enhance clinical excellence. This platform may allow mental health professionals to collaborate and share expertise, ultimately leading to better user outcomes. By having a unified platform, mental health professionals may also be able to provide more comprehensive and coordinated care to users.

Further, in one exemplary embodiment of the present disclosure, the system 101 and method for screening mental health may be designed to cater to different types of users and business models such as B2C (Business to Consumer), B2B (Business to Business), B2G (Business to Government) and A la carte model. Therefore, a single system 101 may cater to all these different business models and allows users to use the system as per their requirements. The user may be from all sectors of the society, including but not limited to, students, older age people, professionals, caregivers and care providers. Further, the system 101 may comprise enhanced solution to prioritize the inclusion of features that enable comparative analysis between users, allowing to identify variations in stress levels and potentially uncover factors contributing to higher or lower stress. By implementing group mapping functionality, visualizing and analyzing screening of multiple users and providing result data in a more comprehensive manner, enabling to identify commonalities or differences among various user groups.

In some embodiment of the present disclosure, the system 101 may act as a preventive profiling tool for the users to screen about their mental health way before developing any acute or chronic situations.

Some embodiments of the system and method may be configured to create API’s, which can be referred or utilize by other systems irrespective of their underlying technologies and implementation, for storing all those network data in the event of usage of this screening tool on the mental health.

Some embodiments of the system and method may be configured to group one or more external systems and logs of attempting answering to the first and the second set of questionnaires for all complex process-oriented subsystems which can be generated, with minimal computation costs.

Some embodiments of the system may have capability to compare a predetermined set of values or data to be compared with the user input values or data.

Some embodiment of the present system may provide notification to the plurality of external systems.

Although implementations for a system and a method for screening tool for mental health using machine learning and AI technology have been described in language specific to structural features and/or methods, it is to be understood that the appended description is not necessarily limited to the specific features or methods described. Rather, the specific features and methods are disclosed as examples of implementations for real-time usage of the screening tool for mental health.

We claims:

1. A method for screening mental health using a screening tool, the method comprising:
a processor configured for:
providing (Step 501) a set of questions to a user/subject by rendering on a user device, wherein the set of questions rendered on the user device are provided by a questionnaire module;
receiving (Step 502) natural language reply to each question in the set of questions, wherein the natural language replies are analyzed using a natural language processing module;
assigning (Step 502) a weightage to each question in the set of questions using the natural language processing module;
generating (Step 504) an Emotional Wellness Index (EWI) for the user, by an assessment module based on the weightage assigned, wherein the Emotional Wellness Index (EWI) is generated for at least one domain selected from domestic life domain, situational/circumstantial domain, nature of the user domain, professional life domain, and clinical domain; and
generating (Step 505) a report based on EWI, wherein a report generation module renders the level of distress to the user based on EWI and specific to which domain.

2. The method as claimed in claim 1, wherein the screening for mental health further comprises capturing (Step 502) facial and behavioral expression of the user by an expression capturing module while replying to each question.

3. The method as claimed in claim 2, wherein assigning the weightage further comprises amending (Step 502) the weightage by an Artificial Intelligence (AI) module based on the expression captured by the expression capturing module.

4. The method as claimed in claim 1, further comprises sending (Step 503) another set of questions to the user, based on the weightage assigned to each question, in the previous set of questions.

5. The method as claimed in claim 4, further comprises determining or selecting questions (Step 503) from the questionnaire module to form another set of questions by the AI module.

6. The method as claimed in claim 5, further comprises updating the questions in the questionnaire module via machine learning based on the historical data, and as number of subjects or users screened using the screening tool.

7. The method as claimed in claim 1, further comprises repeating step 501 to step 503 until questions about domestic life domain, situational /circumstantial domain, nature of the user domain, professional life domain, and clinical questions domain are covered.

8. The method as claimed in claim 1, wherein generating (step 505) the report based on EWI comprises identifying varying levels of distress related to specific domains, indicating the extent of distress in relation to the underlying causes.

9. The method as claimed in claim 1, further comprises recommending AI based therapy, or human interaction-based therapy for the reducing the stress based on the distress level of the user and the specific domain.

10. The method as claimed in claim 1, further comprises automated triaging, by referring users to one of psychologist, psychiatrist, any trained person and a combination thereof, based on the distress level of the user and the specific domain.

11. A system for screening of mental health using a screening tool, the system comprises:
a user devices configured to render a set of questions provided by the questionnaire module;
a natural language processing module configured to quantify the replies by assigning a defined weightage;
an AI module configured select questions from the questionnaire module to form another set of questions based on the weightage assigned to each question;
an expression capturing module is configured to capture the facial and other behavioral expression of the user while replying to each of the questions; assessment module configured to generate an Emotional Wellness Index (EWI) for each of the specific domain including domestic life, situational/circumstantial, nature of the user, professional life, and clinical, wherein the (EWI) is based on the weightage assigned to each reply by the natural language processing module and the AI module; and
a report generation module configured to render the level of distress to the user.
